(19) United States
(12) Patent Application Publication
(10) Pub. No.: US 2011/0319331 A1
(43) Pub. Date: Dec. 29, 2011

(54) NOVEL USES OF FIBRINOGEN

(75) Inventors: Caroline Teboul, Boulogne-Billancourt (FR); Bruno Padrazzi, Paris (FR)

(73) Assignee: Laboratoire Francais Du Fractionnement Et Des Biot, Les Ulis (FR)

(21) Appl. No.: 13/254,565

(22) PCT Filed: Mar. 5, 2010
(86) PCT No.: PCT/FR2010/050380

§ 371 Date: Sep. 2, 2011

(30) Foreign Application Priority Data
Mar. 6, 2009 (FR) 0951440

(51) Int. Cl. A61K 38/36; A61P 7/04

(52) U.S. Cl. 514/13.6

(57) ABSTRACT

The invention relates to the use of fibrinogen for manufacturing a medicinal product for use in the prevention or treatment of severe acute haemorrhage, the medicinal product being intended for administration in an amount equal to at least 4.5 g of fibrinogen in a single dose.

### FIELD OF THE INVENTION

The present invention relates to the area of the treatment of severe acute haemorrhages, including but non-limited to post-partum haemorrhages, post-traumatic and surgical (peri-operative) severe acute haemorrhages.

### PRIOR ART

Severe acute haemorrhages (SAH) are defined by rapid loss—in a few hours—of at least 20 to 30% of an individual's blood volume. The principal situations of SAH occur notably in obstetrics (post-partum haemorrhages essentially), in traumatology and in surgery, but are not limited to these situations.

Disorders of coagulation (coagulopathies) are frequent in the SAH situation and are linked directly or indirectly to the haemorrhage. Among the clotting factors, the plasma fibrinogen level is chronologically the first to drop and reach critical values (Hippala et al., 1995, Anesth Analg, Vol. 81: 360-365; Torrielli et al., 1988, Rev Fr Gynecol Obstet, Vol. 83: 7-9). These threshold values are classically between 0.5 and 1 g/l and correspond to the concentrations permitting satisfactory blood clotting to be obtained, in a stable non-haemorrhagic situation. The mechanisms involved in lowering of the fibrinogen level are: 1/direct losses, 2/dilution by the volume replacement solutions, and 3/consumption of fibrinogen for clot formation.

Besides the fact that fibrinogen is the first clotting factor whose blood level drops, it has been shown that lowering of the fibrinogen level is a factor of poor clinical prognosis, and this lowering is associated with a more severe course for patients with decreasing fibrinogen concentration (Charbit et al., 2006, Journal of Thrombosis and Haemostasis, Vol. 5: 266-273; Karlsson et al., 2008, Transfusion, Vol. 48 (10): 2152-2158).

The conditions for management of an SAH, in an emergency, are adapted to each individual case but have common objectives, including the treatment of clotting disorders when they exist, which is frequent. At present, the administration of fibrinogen is recommended when the assay of plasma fibrinogen reaches the aforementioned critical values. This replacement approach aims to correct situations of constitutional coagulopathies, said coagulopathies often being at the stage of disseminated intravascular coagulation (DIVC). This replacement approach means there is a delay as it is dependent on the time taken to obtain the results of repeated laboratory tests, which it sometimes takes a long time to obtain (close to 1 hour) in order to diagnose an acquired fibrinogen deficiency and document its course over time. Moreover, according to this replacement approach, the administration of fibrinogen is repeated over time as it is determined by the results of repeated determinations of fibrinogen. The dose of fibrinogen can be administered in several infusions, each being determined by the ratio of the quantified deficiency of fibrinogen to the threshold fibrinogen blood value to be reached. The fibrinogen is administered slowly at a recommended rate of less than 5 ml/minute, which is inappropriate in an emergency.

When the circulating fibrinogen level is below 1 g/L, exogenous fibrinogen is administered in order to permit the formation of a normal clot. The amount of exogenous fibrinogen supplied varies from 2 to 4 grams, the amount required being calculated case by case and in real time as a function of the results of the successive assessments of the coagulation parameters described above. More precisely, the amount of fibrinogen to be injected is calculated conventionally according to the following formula: Amount of exogenous fibrinogen (in grams)=[circulating fibrinogen level required (in g/L)]−[circulating fibrinogen level before treatment (in g/L)]×0.04×[patient's weight (in kg)]. Thus, the amounts of fibrinogen to be administered successively during the treatment are calculated as a function of the assessments of the coagulation parameters, also successive over time. It should be noted that accurate calculation of the amount of exogenous fibrinogen to be administered is considered to be essential, in order to avoid an excess of circulating fibrinogen, for example a circulating fibrinogen level greater than 5 g/L, which may cause thromboses.

We may also mention the administration of a platelet concentrate as a means of treatment additional to the means of treatment described above, in particular in situations in which bleeding persists despite the supply of fibrinogen.

The protocols for treatment of post-partum haemorrhages that are currently applied are satisfactory overall. However, taking into account the high risk of mortality or morbidity associated with these severe acute haemorrhages, there is a constant need in the state of the art for technical solutions that are alternative or improved relative to the existing solutions.

There is a need to find alternative or improved means for treating other types of severe acute haemorrhages, including severe haemorrhages caused by surgical procedures or by violent physical trauma. The other severe acute haemorrhages have aspects in common with post-partum haemorrhages, including disorders of homeostasis.

### SUMMARY OF THE INVENTION

The present invention relates to the use of fibrinogen for manufacturing a medicinal product used in the treatment of severe acute haemorrhage, said medicinal product providing early, rapid supply of a sufficient amount of fibrinogen for restoring the clotting capacity of the blood, regardless of the initial fibrinogen level. For example, an amount equal to at least 4.5 g may be administered in a duration of administration of less than 30 minutes.

According to the invention, severe acute haemorrhages include, without being limited to these areas, post-partum haemorrhages, peri-operative haemorrhages and post-traumatic haemorrhages.

The fibrinogen that is administered applies to any fibrinogen, whatever its origin and its nature.

The invention also relates to pharmaceutical compositions, preventive or curative, comprising fibrinogen, which are specially adapted for application of the aforementioned uses and methods.

### DESCRIPTION OF THE DRAWINGS

FIG. 1 illustrates the temporal succession of steps of the experimental protocol in vivo in pigs. The horizontal line represents time. The numbered vertical bars represent successive steps of the protocol. 1: Step of anaesthesia and instrumentation of the animals. 2: Taking the baseline measurements of the parameters investigated, respectively HR (heart rate), MAP (mean arterial pressure), PAP (pulmonary arterial pressure), PCWP (pulmonary central wedge pressure), CVP (central venous pressure), ROTEM®, standard tests: coagulation, haemoglobin, haematocrit, platelets. 3: Step of performing haemodilution, during which 60% of the blood volume is taken, and is replaced with an HES 130/0.4 composition (Voluven®), with the aim of reaching a value of MCF below 40 mm. 4: Step of re-transfusion of the washed red cells, with the aim of reaching a haemoglobin value of 5 to 6 g/dl. 5: Performing the step of bone injury. 7: Performing the measurements of the parameters HR, MAP, PAP, PCWP, ROTEM®, tests for standard coagulation, haemoglobin, haematocrit, platelets and for blood loss at respective times of 15 minutes, 1 hour, 2 hours and 4 hours following administration of the medicinal product (fibrinogen at different doses and placebo composition). 9: Performing the step of hepatic injury. 9: Final step of experiment, 2 hours after hepatic injury, during which measurements are taken of the parameters HR, MAP, PAP, CVP, PCWP, ROTEM, standard tests for blood clotting, haemoglobin, haematocrit, platelets and blood loss.

FIG. 2 shows the results of measurement of the INTEM coagulation time (CT) by the ROTEM® method. FIG. 2, top, shows the results obtained with animals that received, respectively: (i) a placebo composition, represented by filled diamonds, (ii) 37.7 mg/kg of human fibrinogen, represented by filled squares, (iii) 75 mg/kg of human fibrinogen, represented by filled black triangles and (iv) 150 mg/kg of fibrinogen, represented by inverted grey triangles. FIG. 2, bottom, shows the results obtained with animals that received, respectively: (i) a placebo composition, represented by filled diamonds, (ii) 300 mg/kg of human fibrinogen, represented by filled squares, (iii) 450 mg/kg of human fibrinogen, represented by filled grey triangles and (iv) 600 mg/kg of fibrinogen, represented by inverted grey triangles. On the ordinate: coagulation time (CT), expressed in seconds. On the abscissa, the various successive steps of the experiment in vivo, from left to right respectively: (i) after instrumentation and before treatment of the animals, (ii) after haemodilution, (iii) 15 minutes after administration of the medicinal product, (iv) 1 hour after administration of the medicinal product, (v) 2 hours after administration of the medicinal product, (vi) 4 hours after administration of the medicinal product and (vii) 2 hours after performing the hepatic injury or before death.

FIG. 3 shows the results of measurement of INTEM maximum clot firmness (MCF) by the ROTEM® method. FIG. 3, top, shows the results obtained with animals that received, respectively: (i) a placebo composition, represented by filled diamonds, (ii) 37.7 mg/kg of human fibrinogen, represented by filled squares, (iii) 75 mg/kg of human fibrinogen, represented by filled black triangles and (iv) 150 mg/kg of fibrinogen, represented by inverted grey triangles. FIG. 3, bottom, shows the results obtained with animals that received, respectively: (i) a placebo composition, represented by filled diamonds, (ii) 300 mg/kg of human fibrinogen, represented by filled squares, (iii) 450 mg/kg of human fibrinogen, represented by filled grey triangles and (iv) 600 mg/kg of fibrinogen, represented by inverted grey triangles. On the ordinate: maximum clot firmness (MCF), expressed in millimetres. On the abscissa, the various successive steps of the experiment in vivo, from left to right respectively: (i) after instrumentation and before treatment of the animals, (ii) after haemodilution, (iii) 15 minutes after administration of the medicinal product, (iv) 1 hour after administration of the medicinal product, (v) 2 hours after administration of the medicinal product, (vi) 4 hours after administration of the medicinal product and (vii) 2 hours after performing the hepatic injury or before death.

FIG. 4 shows the results of measurement of maximum clot firmness (MCF) PLASMA EXTEM (modified FibTEM) by the ROTEM® method. FIG. 4, top, shows the results obtained with animals that received, respectively: (i) a placebo composition, represented by filled diamonds, (ii) 37.7 mg/kg of human fibrinogen, represented by filled squares, (iii) 75 mg/kg of human fibrinogen, represented by filled black triangles and (iv) 150 mg/kg of fibrinogen, represented by inverted grey triangles. FIG. 4, bottom, shows the results obtained with animals that received, respectively: (i) a placebo composition, represented by filled diamonds, (ii) 300 mg/kg of human fibrinogen, represented by filled squares, (iii) 450 mg/kg of human fibrinogen, represented by filled grey triangles and (iv) 600 mg/kg of fibrinogen, represented by inverted grey triangles. On the ordinate: maximum clot firmness (MCF), expressed in millimetres. On the abscissa, the various successive steps of the experiment in vivo, from left to right respectively: (i) after instrumentation and before treatment of the animals, (ii) after haemodilution, (iii) 15 minutes after administration of the medicinal product, (iv) 1 hour after administration of the medicinal product, (v) 2 hours after administration of the medicinal product, (vi) 4 hours after administration of the medicinal product and (vii) 2 hours after performing the hepatic injury or before death.

FIG. 5 shows the results of measurements of plasma fibrinogen concentration. The curves show the results obtained with animals that received, respectively: (i) a placebo composition, represented by filled diamonds, on the bottom curve of FIG. 5, (ii) 37.5 mg/kg of human fibrinogen, represented by filled squares on the curve immediately above the preceding curve, (iii) 75 mg/kg of human fibrinogen, represented by filled black triangles on the curve immediately above the preceding curve, (iv) 150 mg/kg of fibrinogen, represented by inverted grey triangles on the curve immediately above the preceding curve, (v) 300 mg/kg of human fibrinogen, represented by filled squares on the curve immediately above the preceding curve, (vi) 450 mg/kg of human fibrinogen, represented by filled grey triangles on the curve immediately above the preceding curve, and (vii) 600 mg/kg of fibrinogen, represented by grey triangles on the curve immediately above the preceding curve and which is also the top curve in FIG. 5. On the ordinate: plasma fibrinogen concentration, expressed in mg/dl. On the abscissa, the various successive steps of the experiment in vivo, from left to right respectively: (i) after instrumentation and before treatment of the animals, (ii) after haemodilution, (iii) 15 minutes after administration of the medicinal product, (iv) 1 hour after administration of the medicinal product, (v) 2 hours after administration of the medicinal product, (vi) 4 hours after administration of the medicinal product and (vii) 2 hours after performing the hepatic injury or before death.

FIG. 6 shows the results of measurements of INTEM maximum clot firmness (MCF) by the ROTEM® method as a function of the fibrinogen dose administered to the animals. On the ordinate: value of maximum clot firmness (MCF), expressed in millimetres. On the abscissa, concentration of human fibrinogen, expressed in mg/dl.

FIG. 7 shows measurements of blood loss and of coagulation capacity. On the ordinate: value of blood loss and value of clot size, expressed in ml/kg. In FIG. 7, the respective values of clot size (“clot”) and of blood loss (“liquid”) are represented by adjacent bars, for each of the increasing concentrations of human fibrinogen administered to the animals and for the placebo composition. From left to right in FIG. 7, each pair of adjacent bars (clot/liquid) represents the respective values for (i) 37.5 mg/kg of fibrinogen, (ii) 75 mg/kg of fibrinogen, (iii) 150 mg/kg of fibrinogen, (iv) 300 mg/kg of fibrinogen, (v) 450 mg/kg of fibrinogen, (vi) 600 mg/kg of fibrinogen and (vii) the placebo composition.

### DETAILED DESCRIPTION OF THE INVENTION

Surprisingly, it was shown according to the invention that in the treatment of severe acute haemorrhages, even in the absence of a known fibrinogen deficiency, early and rapid administration of a single large dose of fibrinogen is able to control severe acute haemorrhage and prevent its progression to uncontrollable haemorrhage.

According to the invention, early and rapid administration of a large dose of exogenous fibrinogen makes it possible to interrupt, in the case of severe acute haemorrhage, the progression of an incipient, occult coagulopathy, largely consisting of a progressive decrease in the clotting capacity of the blood, itself caused primarily by the decrease in endogenous fibrinogen, to a constitutive coagulopathy, which can give rise to uncontrollable haemorrhage.

The administration of exogenous fibrinogen, in the conditions specified in this invention, thus constitutes both a treatment of severe acute haemorrhage and prevention of its harmful progression to uncontrollable haemorrhage.

According to the invention, a predetermined amount of exogenous fibrinogen makes it possible to control a severe acute haemorrhage without the need to perform a prior determination of the circulating fibrinogen level. In fact the principle of the treatment according to the invention is not to make up for a fibrinogen deficiency, i.e. supply an amount of fibrinogen based on a threshold blood value predetermined as being critical for attaining a target blood value that is presumed to be effective, but to restore the clotting capacity of the blood as quickly as possible.

Now, taking into account the fibrinogen assay for determining the need to administer fibrinogen, and for determining the amount of fibrinogen that must be administered, means that the results of measurement of the blood fibrinogen level must be available to the medical team. It should be pointed out that the results of measurement of the blood fibrinogen level generally are not available until at least 45 minutes to 1 hour after taking the blood sample, even in an emergency situation. This loss of time in administration of the treatment of a therapeutic emergency can mean that the value of the patient's blood fibrinogen level, at the moment when the measurement results are available, is below the aforementioned threshold value, which can in certain cases significantly affect the patient's chances of survival.

Now, it was shown in the invention that the administration of fibrinogen was effective without needing to know the initial fibrinogen blood level beforehand. It has therefore been shown that an SAH can be treated by early administration of fibrinogen, without prior measurement of the blood level, once a clinical diagnosis of severe acute haemorrhage has been made.

It has thus been shown according to the invention that fibrinogen can be administered to patients with a severe acute haemorrhage with a single amount of fibrinogen, in order to restore the clotting capacity of the blood immediately, i.e. without needing to adjust the amount of fibrinogen as a function of the patient to be treated.

In particular, it was shown according to the invention that severe acute haemorrhages can be treated successfully by administering a predetermined amount of exogenous fibrinogen, without the need to perform a prior determination of the circulating fibrinogen level.

Surprisingly, it was shown according to the invention that early, rapid administration, to a patient with a severe acute haemorrhage, of a single large dose of fibrinogen, without determining the circulating fibrinogen level prior to administration, makes it possible to improve or prevent aggravation of irreversible haemostasis disorders that said patient may experience, and moreover without causing thrombosis.

Moreover, it has also been shown according to the invention that a severe acute haemorrhage can be treated by early and rapid administration of a single large dose of fibrinogen, in an amount equal to at least 4.5 g, i.e. without the need to perform several sequential administrations of exogenous fibrinogen throughout the period of treatment and/or follow-up of the patient, associated with adjustment of the amount of fibrinogen as a function of the fibrinogen levels.

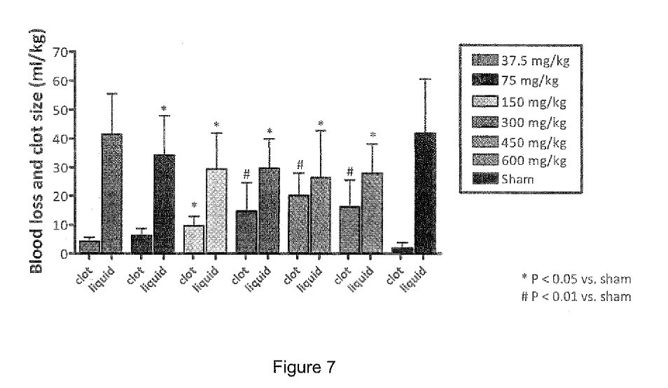

The examples illustrate an animal study in which a range of doses of fibrinogen in the range from 37.5 mg/kg to 600 mg/kg was administered, according to a model of haemodilution caused by a traumatic shock with a 60% loss of blood volume, which is then replaced with a 6% solution of hydroxyethyl starch (HES). The results show that the coagulation time and maximum clot firmness are significantly affected by the haemodilution. The administration of fibrinogen induces a dose-dependent increase in all the measurements of clot firmness. The results in the examples show that treatment of animals that have undergone a haemorrhage with a fibrinogen dose of 50 mg/kg completely restores the values of maximum clot firmness within 15 minutes after the end of fibrinogen infusion, and this effect is maintained until the end of the experiment. With larger doses of fibrinogen, a plateau is reached for the EXTEM MCF values (ROTEM® test). Conversely, with increasing doses of fibrinogen, the INTEM MCF values (ROTEM® test: —platelet-independent coagulation) continue to increase, as shown in FIG. 6. The results in the examples show that thrombin production in animals to which fibrinogen has been administered does not differ from thrombin production in the control group of animals. Statistical analysis of blood loss shows a significant dose-response effect (p=0.02), which indicates a decrease in total blood loss with increasing doses of fibrinogen up to 400 mg/kg, a plateau being reached for a fibrinogen dose of 600 mg/kg, as is shown in FIG. 7. The results in the examples show that the fibrinogen concentration obtained in the animals that received 300 mg/kg of fibrinogen was near the baseline value obtained in control animals that had not undergone haemorrhage. The fibrinogen concentration in the animals that were treated with 400 mg/kg of fibrinogen was even higher than the same group of control animals.

Importantly, it was shown in the examples that, even with high doses of fibrinogen, no sign of hypercoagulation was observed after analysis of the biological or histological parameters.

The results in the examples confirm that clot firmness is affected by low fibrinogen levels, the low fibrinogen levels being caused in the experimental model used by a dilution coagulopathy. The results in the examples confirm the key role of fibrinogen in this type of coagulopathy. The role of fibrinogen is further confirmed by the fact that the single administration of fibrinogen has an intrinsic potential to correct the effects of the coagulopathy, in a dose-dependent manner.

Similarly, the results in the examples show that at least complete restoration of the fibrinogen concentration to the normal levels (ROTEM® test, EXTEM MCF) or even to levels slightly above normal (INTEM MCF and clot weight) is necessary for optimum coagulation to be re-established.

As already mentioned, it is remarkable that even at high doses of fibrinogen, no sign of potential hypercoagulation has been observed.

Thus, the results in the examples show that the administration of a single large dose of fibrinogen, without determining the circulating fibrinogen level prior to administration, makes it possible to prevent aggravation of haemostasis disorders, moreover in complete safety for the patient since, completely surprisingly, no potential effect of hypercoagulation is observed, even with high doses of fibrinogen.

Moreover, it was shown that the administration of a single high dose of fibrinogen according to the invention did not induce a hypersensitivity reaction (measurement of plasma histamine levels). The plasma histamine levels remained stable, for all fibrinogen doses (37.5 mg/kg to 600 mg/kg).

Moreover, the plasma concentrations of TNF-α were reduced or remained stable after administration of fibrinogen. Finally, endothelin-1 has never been detected. Taken together, these results confirm the harmlessness for the patient of the rapid administration of fibrinogen in a single large dose for treating severe acute haemorrhage.

The present invention relates to the use of fibrinogen for manufacturing a medicinal product for use in the treatment of a severe acute haemorrhage, said medicinal product being intended for administration in an amount equal to at least 4.5 g of fibrinogen, in a single dose.

The invention also relates to fibrinogen for use in the treatment of a severe acute haemorrhage, intended for parenteral, preferably intravenous, administration.

The invention also relates to a method for preventing or treating a severe acute haemorrhage, comprising a step of administration of fibrinogen in an amount equal to at least 4.5 g.

It was shown that a single step of administration of the predetermined amount at least equal to 4.5 g of fibrinogen was sufficient for preventing or treating severe acute haemorrhages.

It was shown in the examples that the administration of a predetermined amount at least equal to 4.5 g of fibrinogen is effective and does not lead to any undesirable effect when the fibrinogen is administered rapidly. In particular, in the experimental model of the pig illustrated in the examples, an amount of 600 mg/kg of fibrinogen could be administered within a duration of the administration step of 30 minutes. Transposed to humans, the results in the examples show that a dose of 6 g of fibrinogen can be administered very rapidly, within a duration of the administration step of less than 30 minutes.

According to the invention, a duration of the step of administration of fibrinogen of less than 30 minutes includes durations of less than 29, 28, 27, 26, 25, 24, 23, 22, 21, 20, 19, 18, 17, 16, 15, 14, 13, 12, 11, 10, 9, 8, 7, or 6 minutes.

In the case specifically illustrated in the examples, the step of administration of an amount of 6 g of fibrinogen is of five minutes, without causing an undesirable effect for the patient.

According to the invention, a duration of the step of administration of fibrinogen of less than 30 minutes includes a duration of the administration step greater than 1 minute.

With the use of fibrinogen according to the invention, obligatory recourse to determination of the patient's circulating fibrinogen level prior to the administration of fibrinogen is avoided, which constitutes an important technical advantage for preventing or treating clinical emergencies in patients, for whom rapid preventive or therapeutic intervention can be decisive for the patient's chances of survival.

Thus, the use of fibrinogen according to the invention is further characterized in that the medicinal product is intended for administration in an amount equal to at least 4.5 g of fibrinogen in a single dose, without checking or determining the circulating fibrinogen level prior to its administration to the patient. Accordingly, the method of prevention or treatment according to the invention can moreover be characterized in that it does not comprise a step of checking or determining the circulating fibrinogen level, prior to the step of administration of fibrinogen.

Moreover, the use of fibrinogen according to the invention is further characterized in that the medicinal product is intended for administration in an amount equal to at least 4.5 g of fibrinogen in a single dose, without checking or determining the circulating fibrinogen level subsequent to its administration to the patient. Accordingly, the method of prevention or treatment according to the invention can moreover be characterized in that it does not comprise a step of checking or determining the circulating fibrinogen level, subsequent to the step of administration of fibrinogen.

In addition, absence of determination of the circulating fibrinogen level also represents an economic advantage for the total cost of preventive or curative medical treatment.

The amount of fibrinogen can be at least 4.5 g, 4.6 g, 4.7 g, 4.8 g, 4.9 g, 5 g, 5.1 g, 5.2 g, 5.3 g, 5.4 g, 5.5 g, 5.6 g, 5.7 g, 5.8 g or 5.9 g.

Although the maximum amount of fibrinogen to be administered is not an essential characteristic, said preferred amount of fibrinogen is preferably at most 15 g and especially preferably at most 12 g.

Preferably, the amount of fibrinogen is about 6 g, which includes an amount of fibrinogen in the range from 5.5 g to 6.5 g, including from 5.8 g to 6.2 g.

The use of fibrinogen according to the invention is suitable both for the prevention and for the treatment of certain severe acute haemorrhages.

As an example, the use of fibrinogen according to the invention is suitable for the treatment of post-partum haemorrhages.

In the conventional medical sense, a post-partum haemorrhage is defined as bleeding of a volume of more than 500 mL during the 24 hours following childbirth.

Various risk factors for post-partum haemorrhage are now known, including persistent anaemia, the existence of a congenital haemostasis disorder, situations with risk of uterine atony (e.g. multiparity, uterine overdistension, labour that is long or conversely too quick, existence of chorioamnionitis), situations with risk of anomaly of retraction of the uterus (e.g. placental retention or clots, fibromatous or malformed uterus, placenta accreta), acquired haemostasis disorders, lesions of the birth canal including episiotomy, uterine inversion, uterine rupture or caesarean section.

Thus, in patients for whom there is a significant risk of a post-partum haemorrhage being triggered, the use of fibrinogen according to the invention can be implemented preventively.

However, the use of fibrinogen according to the invention is mainly carried out curatively, in patients who have effectively already started a post-partum haemorrhage.

Similarly, the use of fibrinogen according to the invention is suitable for the prevention and treatment of haemorrhages caused during surgery. In fact, the surgeon can determine in advance, based on his experience, the surgical procedures associated with a high risk of causing a severe acute haemorrhage.

That is why the use of fibrinogen according to the invention is also suitable for the prevention or treatment of severe acute haemorrhages caused during a surgical procedure, i.e. during surgery.

Another situation of severe acute haemorrhage requiring urgent treatment is traumatic haemorrhagic shock, following a severe physical trauma, which is often associated with a sharp drop in volaemia and acute anaemia. In the case of traumatic haemorrhagic shock, the patient's life expectancy is directly related to the volume of blood loss and the promptness of therapeutic management. This is another situation of acute haemorrhage for which a primary aspect of the therapeutic strategy is to re-establish haemostasis.

Thus, according to another aspect, the use of fibrinogen according to the invention is suitable for the treatment of severe acute haemorrhages caused by a physical trauma, also called traumatic haemorrhagic shock.

Quite especially preferably, the fibrinogen consists of human fibrinogen.

In certain embodiments of the use according to the invention, the fibrinogen consists of a purified fibrinogen of natural origin.

In other embodiments of the use according to the invention, the fibrinogen consists of a recombinant fibrinogen. It is possible, for example, to use a recombinant fibrinogen prepared according to any one of the methods described in the PCT international applications published under Nos. WO-207/103447, WO-2005/010178, WO-1996/07728 or WO-1995/022249. Said recombinant fibrinogen can be in the form of a pharmaceutical composition in which the molecules of recombinant fibrinogen are combined with one or more pharmaceutically acceptable excipients.

In the embodiments of the use according to the invention in which the fibrinogen consists of purified fibrinogen of natural origin, said fibrinogen is purified from human plasma and is if necessary combined with one or more pharmaceutically acceptable excipients.

It is possible, for example, to use the purified fibrinogen of natural origin prepared as described in European patent application EP 1 739 093. This is a purified fibrinogen of natural origin that is in the form of a fibrinogen concentrate obtained by a method of purification starting from a solubilized fraction of human plasma which comprises the successive steps of:

a) chromatographic purification comprising the steps of i) charging an anion exchanger of the low base type with said solubilized fraction, previously equilibrated with a buffer of predetermined ionic strength of basic pH, ii) elution of a biological adhesive by increasing the ionic strength of said buffer,

b) separation of Factor XIII from the fibrinogen by adding, to at least a proportion of the eluate of biological adhesive, at least one chemical that precipitates Factor XIII, and recovery of the resultant solution of supernatant of purified fibrinogen, and

c) diafiltration of the solutions of fibrinogen, of biological adhesive and of redissolved FXIII, followed by lyophilization of said solutions.

It is also possible to use purified fibrinogen of natural origin prepared as described in PCT application WO-2005/004901. This is a purified fibrinogen of natural origin that is in the form of a fibrinogen concentrate obtained by a method of purification comprising a step of viral inactivation by thermal treatment of a lyophilizate of cryoprecipitatable proteins of human plasma. The method described in PCT application WO-2005/004901 is characterized notably by adding, before converting the cryoprecipitatable proteins to the form of a lyophilizate, a stabilizing and solubilizing formulation comprising a mixture of arginine, at least one hydrophobic amino acid and trisodium citrate.

It is also possible to use purified fibrinogen of natural origin prepared according to the general method described in European patent application EP 1 739 093 and which comprises a step of viral inactivation of the type described for the method disclosed in PCT application WO-2005/004901.

The purified fibrinogen of natural origin obtained in the form of a fibrinogen concentrate of human plasma by a method selected from the methods described in European patent application EP 1 739 093 or in PCT application WO-2005/004901 possesses the advantage that it comprises a high concentration of human fibrinogen, of the order of 15 g/l to 20 g/l.

This concentrate of human fibrinogen, which can also be denoted “FGT1” in the present description, is particularly suitable for the uses of fibrinogen according to the invention which require the administration of a large amount of fibrinogen at least equal to 4.5 g, preferably in a single dose, on account of the high fibrinogen recovery rate of this type of concentrate. Another advantage of this type of concentrate of human fibrinogen of plasma origin is the low content of other plasma proteins in said concentrate, which greatly reduces the total amount of plasma proteins that are administered to the patient.

An additional advantage is the high degree of viral safety of this type of concentrate of human fibrinogen.

In certain preferred embodiments, the fibrinogen is in the form of a lyophilizate, if necessary in combination with one or more pharmaceutically acceptable excipients.

In certain preferred embodiments, the fibrinogen is in the form of a lyophilizate, if necessary in combination with one or more pharmaceutically acceptable excipients, contained in a container whose internal atmosphere is maintained at a pressure below atmospheric pressure. Advantageously, the interior volume of the container is maintained under a partial vacuum.

Preferably, said lyophilized fibrinogen concentrate is contained in a reconstituting device under vacuum. The fibrinogen solution to be administered to the patient can be prepared extemporaneously by adding, to the bottle under vacuum, the appropriate volume of a suitable solvent, for example water or a sterile and apyrogenic saline solution. In general, the fibrinogen solution obtained after reconstitution from the lyophilized fibrinogen concentrate is stored for at most 7 days, preferably for at most 24 hours, at 25° C. or preferably for at most 6 hours, at 25° C.

In certain embodiments of the use according to the invention, the purified fibrinogen or recombinant fibrinogen is in the form of a pharmaceutical composition in which said fibrinogen is combined with one or more excipients selected from lysine hydrochloride, trometamol, glycine, sodium citrate and sodium chloride. In certain embodiments, said fibrinogen is combined with all of the following excipients: lysine hydrochloride, trometamol, glycine, sodium citrate and sodium chloride. Preferably, for reconstitution of the liquid pharmaceutical composition to be administered, the solvent used consists of water for injection.

In other embodiments of the use according to the invention, the purified fibrinogen or recombinant fibrinogen is in the form of a pharmaceutical composition in which said fibrinogen is combined with one or more excipients selected from arginine hydrochloride, isoleucine, lysine hydrochloride, glycine, and sodium citrate. In certain embodiments, said fibrinogen is combined with all of the following excipients: arginine hydrochloride, isoleucine, lysine hydrochloride, glycine, and sodium citrate. Preferably, for reconstitution of the liquid pharmaceutical composition to be administered, the solvent used consists of water for injection.

A bottle of fibrinogen concentrate “FGT1” to be reconstituted with 100 ml of WFI (water for injection) consists of 1.5 g of fibrinogen, 4 g of arginine, 1 g of isoleucine, 0.2 g of lysine hydrochloride, 0.2 g of glycine and 0.25 g of sodium citrate.

In yet other embodiments of the use according to the invention, the purified fibrinogen or recombinant fibrinogen is in the form of a pharmaceutical composition in which said fibrinogen is combined with one or more excipients selected from human albumin, sodium chloride, arginine hydrochloride, sodium citrate, and sodium hydroxide. In certain embodiments, said fibrinogen is combined with all of the following excipients: human albumin, sodium chloride, arginine hydrochloride, sodium citrate, and sodium hydroxide. Preferably, for reconstitution of the liquid pharmaceutical composition to be administered, the solvent used consists of water for injection.

Preferably, the step of administration of fibrinogen in an amount equal to at least 4.5 g is performed with a medicinal product suitable for injection by the parenteral route, such as the pharmaceutical compositions described above.

In preferred embodiments of the use of the fibrinogen according to the invention, this is performed with a medicinal product suitable for administration by the intravenous (IV) route. Thus, in preferred embodiments of a method of preventive or curative treatment according to the invention, the step of administration of fibrinogen in an amount equal to at least 4.5 g consists of a step of administration by the intravenous route.

In certain embodiments of the use according to the invention, containers under vacuum are used that contain a lyophilized fibrinogen concentrate comprising an amount of 1.5 g of fibrinogen, which means that the contents of four containers are used for administering the desired amount of fibrinogen in a single dose of about 6 g.

In certain preferred embodiments, the reconstituted liquid composition containing human fibrinogen is administered by the intravenous route at a flow rate of administration in the range from 5 mL/minute to 30 mL/minute. Preferably, the reconstituted liquid composition of human fibrinogen is administered by the intravenous route at a flow rate of about 20 mL/minute, which includes the range from 15 mL/minute to 25 mL/minute. A high flow rate of administration of this kind is justified by the urgent need to reduce the problems of homeostasis in patients with a severe acute haemorrhage.

The present invention also relates to a pharmaceutical composition comprising, as active principle, fibrinogen, said pharmaceutical composition being characterized in that it is suitable for the parenteral administration of a single dose comprising an amount of fibrinogen at least equal to 4.5 g, preferably of about 6 g, of fibrinogen.

The present invention is moreover illustrated by, but is not limited to, the following examples.

### EXAMPLES

### Example 1

### Treatment of Post-Partum Haemorrhages

### A. Material and Methods

### Patients

The present study relates to 320 patients who had given birth by the vaginal route or by caesarean section, at more than 27 weeks of pregnancy and presenting a severe post-partum haemorrhage.

The severe post-partum haemorrhage is characterized by a haemorrhage volume greater than or equal to 1000 mL and resistance to 2 lines of uterotonic treatment.

### Concentrate of Human Plasma Fibrinogen

The patients are treated with a single administration of a bolus of 6 g of FGT1, injected intravenously, immediately after reconstitution. The concentrate of human fibrinogen contains 1.5 g of fibrinogen.

### Treatment Protocols

The bolus of 6 g of fibrinogen is administered without first determining the circulating fibrinogen level.

Clinical Observations after Treatment

The volume of blood lost after administration of FGT1 is monitored after administration of the treatment consisting of 6 g of FGT1 administered rapidly by the intravenous route as well as the time elapsed between administration of the FGT1 and the end of haemorrhage. The success of the therapy will be evaluated from absence of recourse to further resources, massive transfusion and death after 12 hours.

### Example 2

### Efficacy in a Haemorrhaciic Pig Model

### A. Material and Methods

### A.1. Study Objective

The study in example 2 consisted of comparing the efficacy of different doses (from 37.5 mg/kg to 600 mg/kg) of fibrinogen compositions (compositions of human fibrinogen concentrate designated “FGT1”) in pigs. The effect on haemostasis and on blood loss was measured in the animals after applying standardized bone and hepatic injuries.

Objectives

The study objectives in example 2 were to determine:
whether the efficacy of a pro-coagulating treatment with different concentrations of fibrinogen induced different effects on haemostasis and blood loss;
whether a single administration of increasing doses of fibrinogen concentrate induced different plasma fibrinogen levels and/or led to different plasma half-lives for the fibrinogen; and
whether the pro-coagulating treatment with fibrinogen concentrate induced a situation of hypercoagulation associated with subsequent thromboembolic complications.

### A.2. Animals

This study was conducted on forty-two healthy pigs aged from 12 to 14 weeks and having a body weight in the range from 25 to 35 kg. The animal model was used in order to determine the dynamics of clot firmness and of blood loss in a situation where fibrinogen is administered after induction of a dilution coagulopathy.

### A.3. Anaesthesia

Pre-medication of the animals was performed with azaperone (4 mg/kg, intramuscular-Stresnil™ injection, Janssen, Vienna, Austria) and of atropine (0.1 mg/kg by intramuscular injection) one hour before the start of the experiment. Induction and maintenance of anaesthesia were performed with propofol (1-2 mg/kg by intravenous injection). For induction of analgesia, piritramide was injected (30 mg, opioid with a half-life of about 4 to 8 hours-Dipidolor™, Janssen, Vienna, Austria). Muscle relaxation was effected by the use of 0.6 mg/kg per hour of rocuronium after endotracheal anaesthesia.

After obtaining endotracheal anaesthesia, the femoral artery, the two femoral veins as well as the subclavian artery were prepared anatomically. The baseline need for liquid replacement (4 mg/kg of body weight) was provided during the test with crystalloids (Ringer lactate solution). Then the following invasive catheters were placed in these vessels:
a catheter of large diameter (double-lumen venous access with a length of 20 cm),
a Swan-Ganz catheter, and
an invasive means of measurement of arterial blood pressure.

Then the initial measurements of the baseline values were taken (time mark No. 2 in FIG. 1).

### A.4. Haemodilution

Following instrumentation as described above, the animals underwent normovolaemic haemodilution with a 6% solution of HES 130/0.4 (Voluven®, Fresenius Co., Bad Homburg, Germany).

Blood was taken from the animals, step by step, using the large-diameter catheters and the blood was replaced with the colloidal solution in 1:1 ratio. To obtain an estimated total blood loss of about 60%, the animals with a weight of for example 30 kg were infused with 1700 ml of 6% HES solution: 130/0.4 (Voluven®, Fresenius Co., Bad Homburg, Germany). After execution of haemodilution, the blood collected was treated in a system of the “cell saver” type (Cats®, Fresenius), and it was concentrated and transfused again, in order to prevent anaemia associated with the haemodynamics. Normovolaemic haemodilution was reached when the resultant coagulopathy reached a value of maximum clot firmness (MCF) below 40 mm, as measured by thromboelastometry (time marks Nos. 3 and 4 in FIG. 1).

### A.5. Standardized Bone Injury

A standardized bone injury was produced by drilling a 3-mm hole in the head of the tibia to a depth necessary for penetrating the bone marrow, five minutes before administering the medicinal product being tested. Five minutes after the bone injury, fibrinogen was measured (time mark No. 5 in FIG. 1), followed by administration of the medicinal product being tested (time mark No. 6 in FIG. 1). The excess blood was removed by suction from the surface of the wound between bone and muscle and was combined in a collecting cell. The time to haemostasis was also determined. When the blood loss due to the bone injury exceeded 500 ml, the haemorrhage was stopped by compression using a standard gauze bandage, to ensure that the pig was stable from the haemodynamic standpoint for the next four hours of observation.

Fifteen minutes after administration of the medicinal product being tested, additional determinations of all the measured parameters were performed (time parameter No. 4). In addition, one hour, two hours and four hours after administration of the medicinal product being tested, all the parameters were measured (time mark No. 7 in FIG. 1).

### A.6. Standardized Hepatic Injury

Four hours after administering fibrinogen, a standardized hepatic injury was produced by making a central incision of the falciform ligament above the central lobe of the liver, using a jig. The resultant hepatic incision has a length of about 8 cm and a depth of about 2 cm (time mark No. 8 in FIG. 1). There was no effect on the catecholamine-mediated circulation. The use of a standardized hepatic injury was chosen so as to demonstrate that the altered coagulation affected mortality negatively.

Two hours after performing the hepatic injury or immediately before suspected death, all the parameters were measured (time mark No. 9 in FIG. 1). Two hours after the hepatic trauma, the surviving animals were euthanized by administering an infusion of potassium to them. According to the results of a previous pilot study, a follow-up evaluation for two hours was considered to be sufficient (Fries et al., 2006, Br. J. Anaesth., Vol. 97 (4): 460-467; Fries et al., 2005, Br. J. Anaesth., Vol. 95 (2): 172-177).

It should be pointed out that the study was blinded.

### A.7. Random Distribution of the Animals (“Randomization”)

Using suitable software, the animals were distributed randomly (randomized) in groups 1 to 6 (1: 37.5 mg/kg, 2: 75 mg/kg, 3: 150 mg/kg, 4: 300 mg/kg, 5: 450 mg/kg and 6: 600 mg/kg).

### A.8. Experimental Protocol

The experimental protocol is illustrated notably in FIG. 1 and the following table, in which the details of the treatment of the 42 pigs included in the study are described.

TABLE 1

| | Volume of the | | |
|---|---|---|---|
| | dose of | | |
| Dose of | fibrinogen | Rate | |
| fibrinogen | (mL/kg) or of | of infusion | Number of |
| (mg/kg) | NaCl | (mL/min)* | animals |
| Placebo (0 | 40 of NaCl | 40 | 6 |
| mg/kg) | | | |
| 37.5 | 2.5 | 2.5 | 6 |
| 75 | 5 | 5 | 6 |
| 150 | 10 | 10 | 6 |
| 300 | 20 | 20 | 6 |
| 450 | 30 | 30 | 6 |
| 600 | 40 | 40 | 6 |

*for a pig of 30 kg.

The fibrinogen was administered by infusion in 30 minutes.

### A.9. Taking Blood Samples and Methods of Analysis

All the blood samples were collected from the femoral artery and the first volume of 5 ml of blood was discarded. The blood samples for the ROTEM® study and analysis of coagulation were collected in 3-ml tubes containing 0.3 ml (0.106 mol/L) of sodium citrate buffer at pH 5.5 (Sarstedt, Nuermbrecht, Germany). The blood samples for the blood cell counts were collected in 2.7-ml tubes containing 1.6 mg of EDTA/ml (Sarstedt, Nuermbrecht, Germany). All the tests were carried out by the same tester. The prothrombin time (PT), thrombin generation time (TGT), partial thromboplastin time (PTT-LA1), fibrinogen concentration, antithrombin (AT) and thrombin-antithrombin (TAT) were determined by standard laboratory methods using appropriate tests from Dade Behring, Marburg, Germany and Amelung coagulometry apparatus (Baxter, United Kingdom). The D-dimer-0020008500® test (Instrumentation Laboratory Company, Lexington, United States) was used for the measurements of D-dimer. The blood cell counts were performed using the Sysmex Poch-100i® counter (Sysmex, Lake Zurich, Ill., United States).

### A.10. Rotary Thromboelastometry (ROTEM®)

ROTEM® is a diagnostic tool of the “point of care” type, with which a coagulation test can be performed on whole blood without requiring time-consuming laboratory testing. Moreover, and in contrast to the standard coagulation tests, with this method it is possible to evaluate information on clot quality, and quite especially on clot firmness.

ROTEM® consists of a modification of the thromboelastography concept that was originally developed by Hartert in 1948. The method consists of a cylindrical sensor, which is immersed in a cuvette containing the blood sample. The sensor is rotated at a degree of 4.75° relative to its longitudinal axis. This movement is altered once the strands of fibrin start to form between the cuvette and the sensor. This inhibition is detected by a change in the light reflection factor, which is detected continuously and transformed to a typical classical curve of the ROTEM® type in which the following parameters are determined:
CT, denoting clotting time, expressed in seconds, which is the time elapsed up to the start of coagulation,
CFT, denoting clot formation time, expressed in seconds, which is the time elapsed between the start of coagulation and the moment when an amplitude of 20 mm is reached,
MCF, denoting maximum clot firmness, expressed in millimetres, which is the maximum amplitude, and
maximum lysis (%), which is the percentage amplitude (MCF), 60 minutes after the start of coagulation.

ROTEM® Tests

Various tests of coagulation activators are available, as detailed below. The tests used in example 2 are the INTEM test and the modified FIBTEM test.
INTEM: when an INTEM test is performed, coagulation is activated intrinsically. The INTEM reagent contains ellagic acid, which is a strong activator of the intrinsic coagulation pathway.
EXTEM: when an EXTEM test is performed, coagulation is activated by the tissue factor.
FIBTEM: the FIBTEM test activates coagulation with the tissue factor. At the same time, the thrombocyte function is altered by adding cytochalasin D. This results in formation of a clot which is induced solely by fibrinogen.

The modified FIBTEM test was used because previous experiments had shown that pig thrombocytes cannot be completely blocked by cytochalasin D. For this reason, the thrombocytes were removed completely from this test by performing an EXTEM test on a plasma sample instead of whole blood (modified FIBTEM).

### A.11. Taking Tissue Samples, Preservation, Preparation of Test Slides and Microscopic Examination

The tissue samples from organs (lungs, heart, kidneys, intestines and liver) were taken at the end of each test after dissection of the animal. The samples were immersed immediately in 10% formalin solution. After dehydration by an ascending sequence of steps employing samples of alcohol, the samples were embedded in paraffin and cut into slices with a thickness of 7 μm. The test slides were stained by classical haematoxylin/eosin staining, and were then examined.

### A.12. Statistical Analysis

The Shapiro-Wilks test was used for verifying normal distribution of the study variables. The presumption of normal distribution was rejected for the following variables: ZVD, WEDGE, SPO2, CT, ALPHA.

For these variables, only non-parametric tests are valid.

Parametric tests: analysis of variance (ANOVA) was used for repeated measurements in order to evaluate differences between the test groups. A significance value of 0.05 was adopted for the groups of effects, the measurements, and measurement of group x.

Multiple comparisons were evaluated against the placebo group using Dunnett's protocol.

A non-parametric test was performed using the Kruskal-Wallis test: all the groups were compared in a single test. A level of statistical significance of 0.05 was adopted.

The Wilcoxon test was compared for each test group, with the placebo group.

Based on the Bonferroni multiple comparison protocol, only P values below 0.0084 (0.05 divided by the number of comparisons (6)) were regarded as statistically significant values.

A Jonckheere-Terpstra test was used in order to evaluate whether total blood loss decreased with increasing doses of fibrinogen. Based on the fact that the distribution of total blood loss is not normal, this non-parametric test for ordered differences in the treatment groups is appropriate (non-parametric statistical methods, Hollander and Wolfe, 1973). The Jonckheere-Terpstra test tests the null hypothesis that the distribution of total blood loss does not differ for groups that received different doses of fibrinogen (control, 37.5 mg/kg, 75 mg/kg, 150 mg/kg, 300 mg/kg, 450 mg/kg and 600 mg/kg, respectively).

The Student test was used in order to compare the parameters specific to the baseline control values after haemodilution.

### B. Results

### B.1. Coagulation Parameters

1) ROTEM® parameters

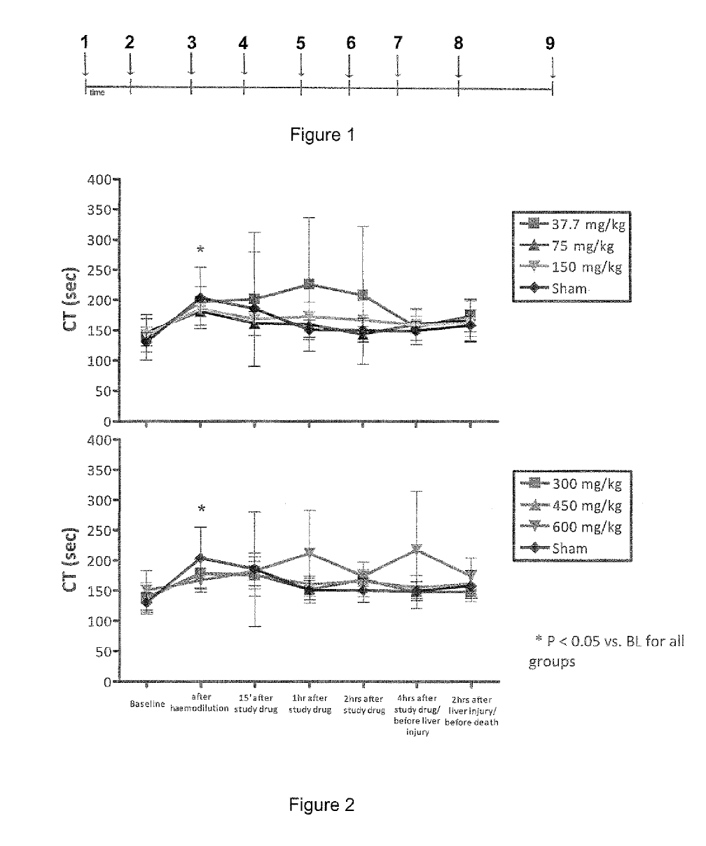

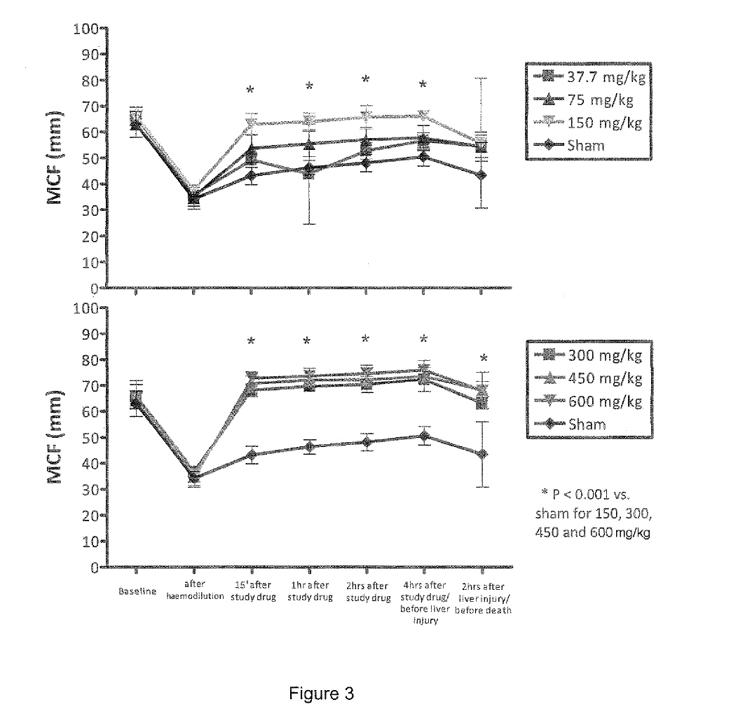

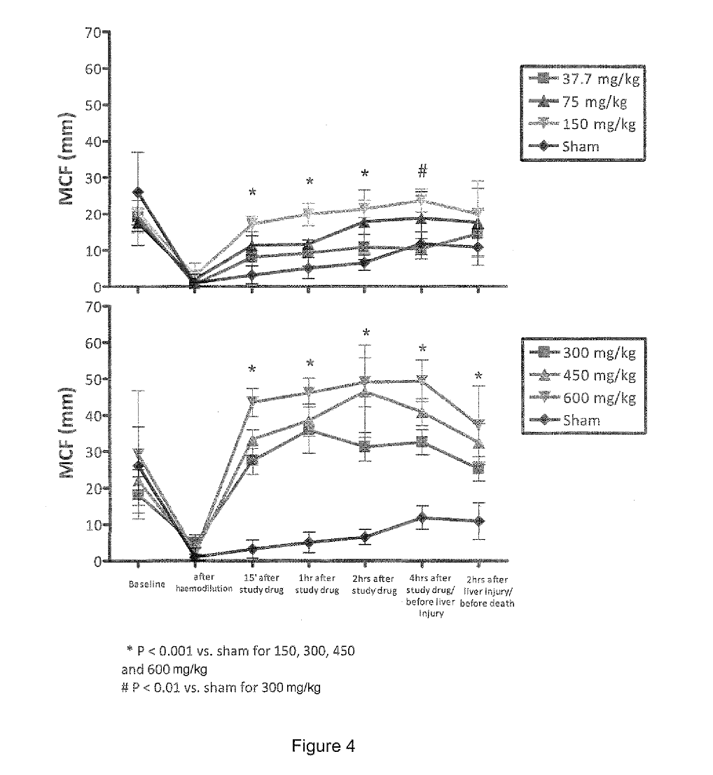

The ROTEM® parameters were significantly affected after haemodilution with Voluven®. The coagulation time increased and the value of maximum clot firmness decreased significantly after infusion of Voluven® (p<0.0001). Similarly, angle α decreased significantly and clot formation time increased significantly (p<0.001). The administration of fibrinogen did not significantly shorten the extended clotting time but it increased MCF significantly (p<0.001 against placebo for the groups corresponding to doses 150 mg/kg to 600 mg/kg, 15 minutes after completion of fibrinogen infusion) and angle α (p<0.05 against placebo for all dosage groups 15 minutes after completion of fibrinogen infusion). Within the context of haemodilution to 60%, the results show that treatment with a dose of 150 mg/kg of fibrinogen is capable of completely restoring the initial baseline MCF values. Larger doses of fibrinogen caused an increase in the INTEM MCF values up to a maximum of 80 mm, the value at which a plateau is reached (FIGS. 2-4 and 6).

2) Standard Coagulation Tests

The value of PT increased significantly from 11.11±0.7 is to 17.43±1.9 is after haemodilution (p<0.0001 against the baseline value or “BL”). After administration of fibrinogen, values of PT increased significantly to 14.54±1.44 s, compared with haemodilution (p<0.0001); however, there was no significant difference between the groups.

The values of aPTT increased significantly from 10.94±3.34 s initially to 21.7±2.72 s after haemodilution (p<0.001).

After administration of fibrinogen, the value of aPTT remained elevated compared with the initial value. There was no difference between the groups, apart from group F which had a significantly increased value of aPTT, compared with the placebo group and the other dosage groups, at all times after administration of the medicinal product.

The value of D-dimer increased significantly compared with the placebo, at time four hours after administering fibrinogen for groups E and F (p<0.01). For groups A, D, E and F, a significant increase in the value of D-dimer was found at the end of the experiment (p<0.05 for group A and p<0.001 for groups D, E and F).

The values of TAT and of thrombin generation (Calatzis method) did not differ from the values of the placebo group.

3) Plasma Fibrinogen Concentration

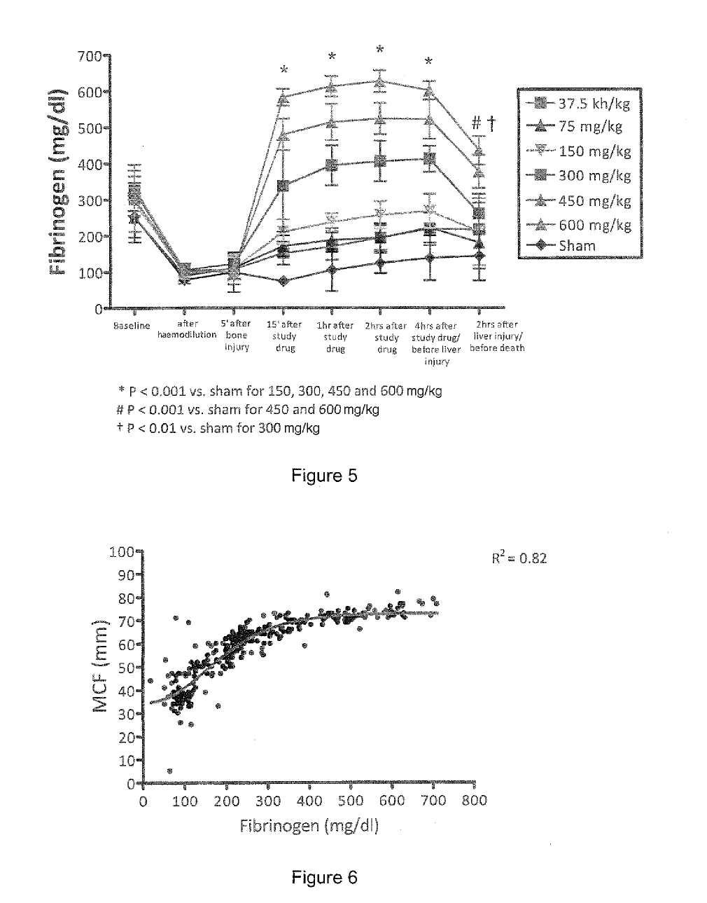

The plasma fibrinogen concentration decreased significantly after haemodilution, compared with the baseline value (p<0.0001) as shown in FIG. 5. All the animals treated with doses of fibrinogen of 150 mg/kg or more showed a significant increase in plasma fibrinogen levels compared with the placebo, at times 15 minutes, 1 hour, 2 hours and 4 hours after treatment (p<0.001).

Group B showed a significant increase in fibrinogen concentration only 15 minutes, 1 hour and 4 hours after treatment (p<0.05). All the groups showed a decrease in fibrinogen levels 2 hours after hepatic injury or just before death. However, the animals treated with doses of 300 mg/kg or more of fibrinogen showed significantly increased fibrinogen levels at time 2 hours, compared with the placebo.

### B.2. Blood Counts

After taking 60% of the total blood volume and replacing the blood with Voluven®, the haemoglobin values dropped significantly to levels between 3-4 g/dl (p<0.0001, relative to the baseline value “BL”). The haematocrit dropped significantly in parallel, to values between 11°/o and 13% (p<0.0001). After re-transfusion of the red blood cells, the haemoglobin and haematocrit values increased significantly to 5-6 g/dl and to 18-20% respectively (p<0.0001 relative to the baseline value “BL” for both parameters).

### B.3. Haemodynamics and Mixed Oxygen Saturation

The venous mixed oxygen saturation increased significantly after haemodilution (p<0.0001 against the baseline value “BL”) and increased again after re-transfusion of red blood cells (p<0.01 after haemodilution). There was a significant increase in venous mixed oxygen saturation (p<0.001), 2 hours after the hepatic injury, or just before the death of the animals, compared with the time after re-transfusion of the red blood cells.

### B.4. Blood Loss

The total blood loss after bone injury and hepatic injury was:
42.12±18.792 ml/kg in the placebo group,
41.55±13.944 ml/kg at 37.5 mg/kg of fibrinogen,
34.30±13.593 ml/kg at 75 mg/kg of fibrinogen,
29.41±12.508 ml/kg at 150 mg/kg of fibrinogen,
29.79±10.155 ml/kg at 300 mg/kg of fibrinogen,
26.59±16.250 ml/kg at 450 mg/kg of fibrinogen, and
28.02±10.325 ml/kg at 600 mg/kg of fibrinogen.

Statistical analysis showed a significant dose-response effect (p=0.02), indicating a reduction of total blood loss for increasing doses of fibrinogen.

The animals that received 150 mg/kg of fibrinogen or higher doses displayed a significant increase in clot forming capacity, as illustrated by a significant increase in size of clot that formed on the surface of the liver after injury (150 mg/kg: p<0.05 against placebo; 300-600 mg/kg: p<0.01 against placebo), as shown in FIG. 7.

### B.6. Histological Examination

Histological examination of the kidneys, intestines, spleen, lungs, heart and liver of the animals did not reveal a microvascular thrombosis of any kind in the vessels.

### CONCLUSION

It is known that, as a result of a massive haemorrhage, the plasma fibrinogen concentration reaches critical levels before any other clotting factor.

The first line of treatment for a severe haemorrhage consists of the administration of crystalloids and colloids in order to maintain normovolaemia. However, colloids and quite especially hydroxyethyl starch (HES) are known to affect fibrin polymerization.

The consequence is reduced resistance of the clot, which can lead to a subsequent blood loss.

The first main result from the study in example 2 is that the administration of fibrinogen is able to effect a dose-dependent reversal of dilution coagulopathy. The ROTEM® measurements clearly showed that maximum clot firmness (MCF) was increased and normalized after administration of fibrinogen.

The values of INTEM showed that administration of 150 mg/kg of fibrinogen was capable of completely restoring the initial MCF values.

The modified FIBTEM test showed the same tendency regarding the MCF values; however, for dosages of 300, 450 and 600 mg/kg, the MCF values increased well beyond the initial levels.

All the animals showed a dose-dependent increase in plasma fibrinogen concentration, which was stable throughout the experiment.

It is important to note that, even if the plasma fibrinogen concentration increased beyond the initial levels, the MCF INTEM values did not follow this increase linearly: at plasma fibrinogen concentrations of 350 mg/dl, the MCF values reached a plateau; a further increase in fibrinogen concentration did not lead to an increase in MCF values.

These results suggest the existence of a safeguarding mechanism which prevents overreaction of the coagulation process in a situation of excess bioavailability of fibrinogen. The presence of platelets seems to be crucial for implementation of this mechanism, since the data for plasma EXTEM (where platelets are practically absent) do not suggest such behaviour: quite especially in the high-dosage groups (300-600 mg/kg), the MCF values increase well beyond the baseline value.

Surprisingly, the results in example 2 show that fibrinogen does not induce a state of hypercoagulation, and does not induce thromboembolic events. In particular, no signs of thromboembolic events were detected, whether macroscopically or microscopically.

Fibrinogen did not affect thrombin production, or TAT.

As far as the applicant knows, the results presented in example 2 are the first to show that fibrinogen doses as high as 12 times the fibrinogen dosage recommended in humans (according to the Austrian Society of Anaesthesiology, Reanimation and Intensive Care Medicine, accessible at the following Internet address: http://www.oeagri.at/dateiarchiv/116/Traumainduziertes %20Gerinnungsmanagement.pdf), can be administered in vivo.

The results in example 2 also showed a reduction of blood loss after administration of fibrinogen and a dose-dependent increase in clot size after hepatic injury.

These results are to be compared with the data concerning an increase in MCF values after treatment with fibrinogen and clearly show that clot forming capacity was improved after administration of fibrinogen. Notably, fibrinogen doses of 150 mg/kg or higher significantly increased clot size.

To summarize, the study in example 2 confirms that the administration of a concentrate of human fibrinogen (FGTW) is able to effect a dose-dependent reversal of dilution coagulopathy. A treatment at a dose of 150 mg/kg is capable of completely restoring the MCF values to the baseline value. The results in example 2 also show that the MCF INTEM values reach a plateau at plasma fibrinogen concentrations of 350 mg/dl. Higher plasma fibrinogen concentrations do not produce a further increase in the MCF values. The administration of fibrinogen significantly reduces blood loss after hepatic injury and produces a dose-dependent increase in coagulation capacity.

These results suggest that even higher doses of fibrinogen are completely harmless in humans, as hypercoagulability has not been detected. Treatment at doses twelve times higher than the recommended dose in humans does not produce any undesirable effect such as thrombosis or pulmonary embolism.

The results in the examples also show that a high dose of fibrinogen can be administered in an administration step of very short duration, without causing an undesirable effect.

As shown in detail below, the results in example 2, when transposed to administration of fibrinogen in humans, indicate that a dose of 6 g of fibrinogen can be administered in a time of the administration step of five minutes, the administration of fibrinogen having the effect of preventing or stopping a haemorrhage, without simultaneously leading to an undesirable effect for the patient. More precisely, the results in example 2 show that the beneficial effects expected on re-establishment of the haemodynamic parameters, without causing any undesirable effect, are obtained by administering, to the pig experimental model illustrated, an amount of fibrinogen of 600 mg/kg with a duration of the administration step of 30 minutes, i.e. a fibrinogen dose of 0.02 g/kg/min. It should be noted that the composition of fibrinogen concentrate that was used in example 2 (composition “FGT1”) has a concentration of human fibrinogen of 15 g/l, or 0.015 g/ml. To obtain a fibrinogen dose of 0.02 g/kg/min, pigs are administered a dose of the aforementioned composition “FGT1” of 1.33 ml/kg/min. Transposed to a human patient with a weight of 60 kg, administration of a dose of composition “FGT1” of 1.33 ml/kg/min comprises administration of composition “FGT1” to said patient according to a rate of administration of 80 ml/min. Thus, for a fibrinogen dose of 6 g to be administered to said human patient, using composition “FGT1”, it is necessary to administer a volume of 400 ml of composition “FGT1” to said patient, at a rate of administration of 80 ml/min. Accordingly, a dose of 6 g of fibrinogen, with the aforementioned rate of administration, can be administered to said human patient in five minutes.

1.-12. (canceled)

13. A method for treating or preventing severe acute haemorrhage in a patient, the said method comprising administering to the patient an amount equal to at least 4.5 g of fibrinogen in a single dose, rapidly with a duration of administration of less than 30 minutes.

14. The method according to claim 13 wherein fibrinogen is administered by the parenteral route, preferably by the intravenous route.

15. The method to claim 13 wherein fibrinogen is administered independently of the initial circulating fibrinogen level of the said patient.

16. The method according to claim 13 wherein the administration of fibrinogen makes it possible to restore the clotting capacity of the blood.

17. The method according to claim 13, wherein the administration of fibrinogen makes it possible to control severe acute haemorrhage and prevent its progression to uncontrollable haemorrhage.

18. The method according to claim 13 for treating severe acute post-partum haemorrhages.

19. The method according to claim 13 for preventing or treating of severe acute haemorrhages during surgery.

20. The method according to claim 13 for treating severe acute post-traumatic haemorrhages.

21. The method according to claim 13 for preventing and treating other severe acute haemorrhages.

22. The method according to claim 13 wherein the fibrinogen is selected from a recombinant fibrinogen and a purified natural fibrinogen.

23. The method according to claim 13 wherein the fibrinogen consists of a fibrinogen concentrate with high viral safety.

24. Pharmaceutical composition comprising fibrinogen as active principle, said pharmaceutical composition being suitable for parenteral administration of a single dose comprising an amount of fibrinogen equal to at least 4.5 g.
